(19) United States
(12) Patent Application Publication
(10) Pub. No.: US 2008/0293714 A1
(43) Pub. Date: Nov. 27, 2008

(54) Tec Kinase Inhibitors

(76) Inventors: Joerg Martin Bentzien, White Plains, NY (US); Brian Nicholas Cook, Danbury, CT (US); Hidenori Takahashi, LaGrangeville, NY (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(21) Appl. No.: 12/095,955

(22) PCT Filed: Dec. 6, 2006
(86) PCT No.: PCT/US06/61649

§ 371 Date: Jun. 3, 2008

Related U.S. Application Data
(60) Provisional application No. 60/743,049, filed on Dec. 20, 2005.

(51) Int. Cl. A61K 31/4162; C07D 495/04; A61K 31/5377; A61P 35/00; A61P 11/00; A61P 29/00; A61K 31/4439

(52) U.S. Cl. 514/234.2; 548/360.5; 544/140; 546/275.7; 514/338; 514/406

(57) ABSTRACT

Disclosed are compounds of formula (I): (F) wherein Ri, R2, X and Ar are defined herein. The compounds of the invention inhibit Itk kinase and are therefore useful for treating diseases and pathological conditions involving inflammation, immunological disorders and allergic disorders. Also disclosed are processes for preparing these compounds and to pharmaceutical compositions comprising these compounds.

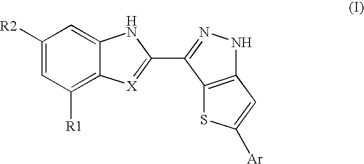

This application claims benefit to U.S. provisional application Ser. No. 60/743,049 filed 12-20-2005.

### TECHNICAL FIELD OF THE INVENTION

This invention relates to compounds of formula (I):

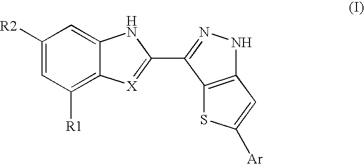

wherein R₁, R₂, X and Ar are defined herein below. The compounds of the invention inhibit Itk kinase and are therefore useful for treating diseases and pathological conditions involving inflammation, immunological disorders and allergic disorders. This invention also relates to processes for preparing these compounds and to pharmaceutical compositions comprising these compounds.

### BACKGROUND OF THE INVENTION

Protein kinases play a critical role in mediating signaling events leading to cellular responses such as activation, growth and differentiation, in response to extracellular signals. Protein kinases transmit their signal by phosphorylating specific residues in a target protein. Protein kinases that specifically phosphorylate tyrosine residues are referred to as protein tyrosine kinases. Protein tyrosine kinases can be divided into two general groups: receptor such as epidermal growth factor (EGF) receptor (S. Iwashita and M. Kobayashi, 1992, Cellular Signalling, 4, 123-132) and cytosolic non-receptor (C. Chan et al., 1994, Ann. Rev. Immunol., 12, 555-592).

Interleukin-2-inducible T cell kinase (Itk), also referred to as T cell-specific kinase (Tsk) and expressed mainly in T-lymphocytes (EMT), is a member of the Tec family of protein tyrosine kinases that also includes Txk, Tec, Btk, and Bmx. Tec family members are characterized by the presence of a pleckstrin-homology domain (PH), a proline rich Tec homology domain (TH) and Sre homology SH3, SH2 and SH1 kinase domains positioned from the N-terminus to the C-terminus respectively (S. Gibson et al., 1993, Blood, 82, 1561-1572; J. D. Siliciano et al., 1992, Proc. Nat. Acad. Sci., 89, 11194-11198; N. Yamada et al., 1993 Biochem. and Biophys Res. Comm., 192, 231-240).

Itk is expressed in T cells, mast cells and natural killer cells. It is activated in T cells upon stimulation of the T cell receptor (TCR), and in mast cells upon activation of the high affinity IgE receptor. Following receptor stimulation in T cells, Lck, a src tyrosine kinase family member, phosphorylates Y511 in the kinase domain activation loop of Itk (S. D. Heyeck et al., 1997, J. Biol. Chem., 272, 25401-25408). Activated Itk, together with Zap-70 is required for phosphorylation and activation of PLC-γ (S. C. Bunnell et al., 2000, J. Biol. Chem., 275, 2219-2230). PLC-γ catalyzes the formation of inositol 1,4,5-triphosphate and diacylglycerol, leading to calcium mobilization and PKC activation, respectively. These events activate numerous downstream pathways and lead ultimately to degranulation (mast cells) and cytokine gene expression (T cells) (Y. Kawakami et al., 1999, J. Leukocyte Biol., 65, 286-290).

The role of Itk in T cell activation has been confirmed in Itk knockout mice. CD4⁺T cells from Itk knockout mice have a diminished proliferative response in a mixed lymphocyte reaction or upon Con A or anti-CD3 stimulation. (X. C. Liao and D. R. Littman, 1995, Immunity, 3, 757-769). Also, T cells from Itk knockout mice produced little IL-2 upon TCR stimulation resulting in reduced proliferation of these cells. In another study, Itk deficient CD4⁺T cells produced reduced levels of cytokines including IL-4, IL-5 and IL-13 upon stimulation of the TCR, even after priming with inducing conditions. (D. J. Fowell, 1999, Immunity, 11, 399-409).

The role of Itk in PLC-γ activation and in calcium mobilization was also confirmed in the T cells of these knockout mice, which had severely impaired IP₃ generation and no extracellular calcium influx upon TCR stimulation (K. Liu et al., 1998, J. Exp. Med. 187, 1721-1727). The studies described above support a key role for Itk in activation of T cells and mast cells. Thus an inhibitor of Itk would be of therapeutic benefit in diseases mediated by inappropriate activation of these cells.

It has been well established that T cells play an important role in regulating the immune response (Powrie and Coffman, 1993, Immunology Today, 14, 270-274). Indeed, activation of T cells is often the initiating event in immunological disorders. Following activation of the TCR, there is an influx of calcium that is required for T cell activation. Upon activation, T cells produce cytokines, including IL-2,4, 5, 9, 10, and 13 leading to T cell proliferation, differentiation, and effector function. Clinical studies with inhibitors of IL-2 have shown that interference with T cell activation and proliferation effectively suppresses immune response in vivo (Waldmann, 1993, Immunology Today, 14, 264-270). Accordingly, agents that inhibit T lymphocyte activation and subsequent cytokine production, are therapeutically useful for selectively suppressing the immune response in a patient in need of such immunosuppression.

Mast cells play a critical roll in asthma and allergic disorders by releasing pro-inflammatory mediators and cytokines. Antigen-mediated aggregation of FcεRI, the high-affinity receptor for IgE results in activation of mast cells (D. B. Corry et al., 1999, Nature, 402, B118-23). This triggers a series of signaling events resulting in the release of mediators, including histamine, proteases, leukotrienes and cytokines (J. R. Gordon et al., 1990, Immunology Today, 11, 458-464.) These mediators cause increased vascular permeability, mucus production, bronchoconstriction, tissue degradation and inflammation thus playing key roles in the etiology and symptoms of asthma and allergic disorders.

Recent published data using Itk knockout mice suggests that in the absence of Itk function, increased numbers of memory T cells are generated (A. T. Miller et al., 2002 The Journal of Immunology, 168, 2163-2172). One strategy to improve vaccination methods is to increase the number of memory T cells generated (S. M. Kaech et al., Nature Reviews Immunology, 2, 251-262).

WO 05/026175 (Aventis) discloses thienopyrazoles which are alleged to possess ITK activity. The compounds disclosed in the present application will posses improved activity over those disclosed by the Aventis application.

All patent and literature documents cited in this application are incorporated by reference in their entirety.

### SUMMARY OF THE INVENTION

It is therefore an object of the invention to provide a compound of the formula (I):

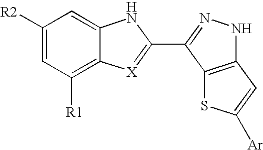

wherein R₁, R₂, X and A are defined herein below.

It is another object of the invention to provide a method of inhibiting the Tec kinase family, including Itk kinase, and methods of treating diseases or conditions related to such kinase activity, by administering to a patient in need thereof a therapeutically effective amount of a compound of the formula (I).

It is yet another object of the invention to provide pharmaceutical compositions and processes of making compounds of the formula (I) as described herein below.

### DETAILED DESCRIPTION OF THE PREFERRED EMBODIMENTS

In it's broadest generic embodiment, the invention provides for a compound of the formula (I):

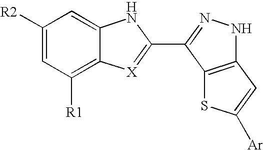

wherein:

X is CH or N;

Ar is chosen from

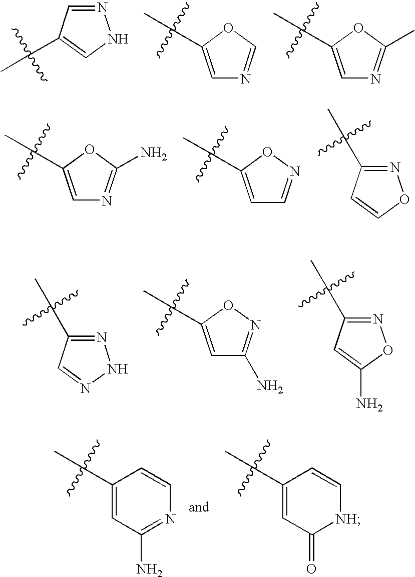

R1 is

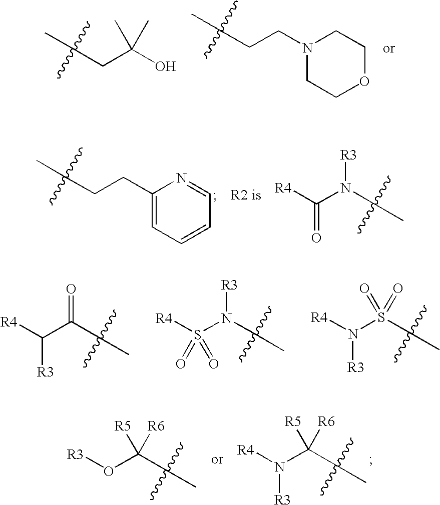

R3, R4 are independently chosen from hydrogen, alkyl, carbocycle, heterocycle C0-5alkyl and heteroaryl wherein each cycloalkyl, heterocycle, aryl and heteroaryl are optionally substituted by C1-5alkyl;

or R3 and R4 optionally combine together to form following rings:

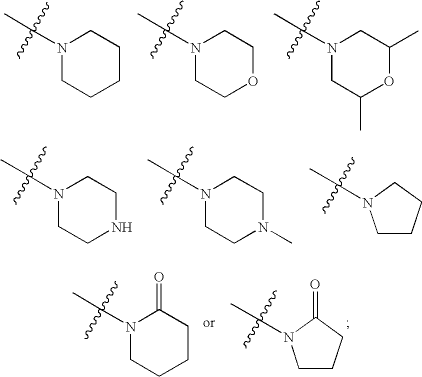

R5, R6 are independently chosen from hydrogen, C1-5alkyl, or optionally combine together to form 3, 4, 5 or 6 membered cycloalkyl ring;

or the pharmaceutically acceptable salts, esters, acids, isomers or tautomers thereof.

In yet another embodiment, there is provided a compound of the formula (I) as described immediately above and wherein:

X is CH;

R3, R4 are independently chosen from hydrogen, C1-5 alkyl, C4-8cycloalkyl, phenyl, naphthyl, morpholinyl, morpholinyl C1-5alkyl, pyrrolidine, pyrrolidinone, benzothiophenyl, benzodioxolyl, quinolinyl, indolyl, thiazolyl, thienyl, furanyl, isoxazolyl, oxazolyl, imidazolyl, thiadiazolyl, pyrazolyl, pyrazinyl and pyridinyl wherein each cycloalkyl, heterocycle, aryl and heteroaryl are optionally substituted by C1-5alkyl; or R3 and R4 optionally combine together to form following rings:

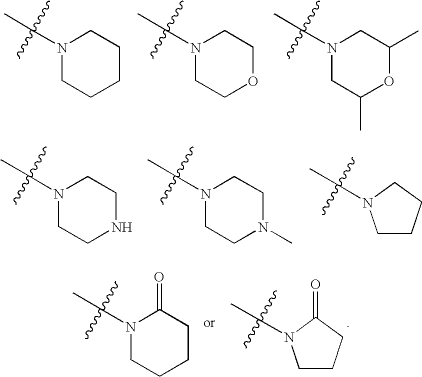

In yet another embodiment, there is provided a compound of the formula (I) as described immediately above and wherein:

R3, R4 are independently chosen from hydrogen, C1-5 alkyl, C4-6cycloalkyl, phenyl, naphthyl, morpholinyl, morpholinyl, morpholinylcl-5alkyl, pyrrolidine, pyrrolidinone, wherein each cycloalkyl, heterocycle and aryl are optionally substituted by C1-5alkyl; or R3 and R4 optionally combine together to form following rings:

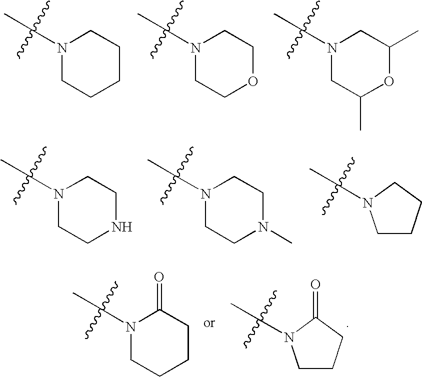

In another embodiment there is provided representative compounds of the invention which can be made in accordance with the general schemes and working examples presented below:

TABLE I
or the pharmaceutically acceptable salts thereof.

| Structure | Name |
|---|---|
| 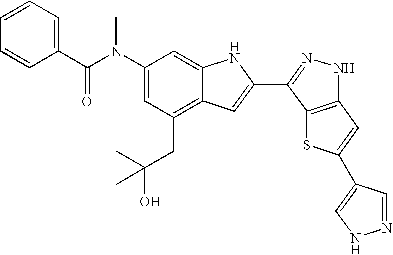 | N-{4-(2-Hydroxy-2-methyl-propyl)-2-[5-(1H-pyrazol-4-yl)-1H-thieno[3,2-c]pyrazol-3-yl]-1H-indol-6-yl}-N-methyl-benzamide |
| 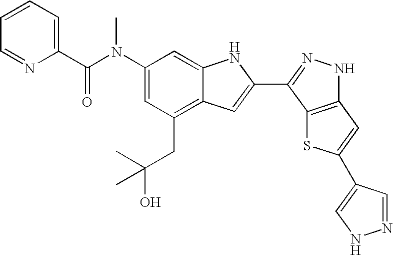 | Pyridine-2-carboxylic acid {4-(2-hydroxy-2-methyl-propyl)-2-[5-(1H-pyrazol-4-yl)-1H-thieno[3,2-c]pyrazol-3-yl]-1H-indol-6-yl}-methyl-amide |
| 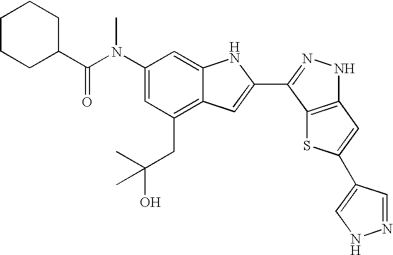 | Cyclohexanecarboxylic acid {4-(2-hydroxy-2-methyl-propyl)-2-[5-(1H-pyrazol-4-yl)-1H-thieno[3,2-c]pyrazol-3-yl]-1H-indol-6-yl}-methyl-amide |
| 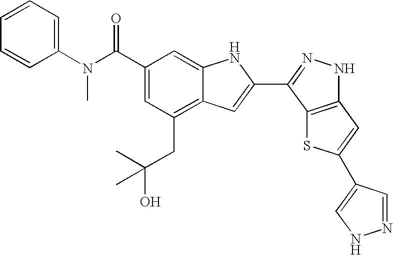 | 4-(2-Hydroxy-2-methyl-propyl)-2-[5-(1H-pyrazol-4-yl)-1H-thieno[3,2-c]pyrazol-3-yl]-1H-indole-6-carboxylic acid methyl-phenyl-amide |
| 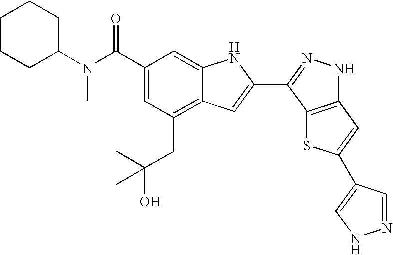 | 4-(2-Hydroxy-2-methyl-propyl)-2-[5-(1H-pyrazol-4-yl)-1H-thieno[3,2-c]pyrazol-3-yl]-1H-indole-6-carboxylic acid cyclohexyl-methyl-amide |
| 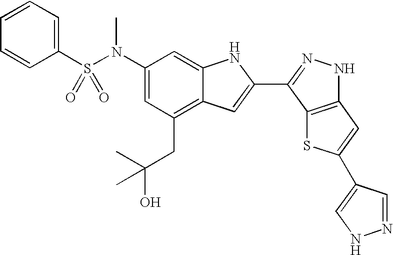 | N-{4-(2-Hydroxy-2-methyl-propyl)-2-[5-(1H-pyrazol-4-yl)-1H-thieno[3,2-c]pyrazol-3-yl]-1H-indol-6-yl}-N-methyl-benzenesulfonamide |
| 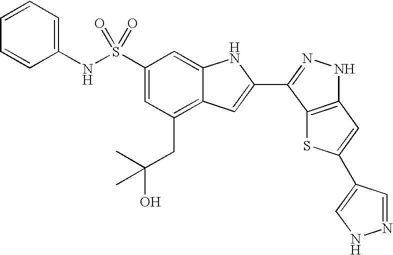 | 4-(2-Hydroxy-2-methyl-propyl)-2-[5-(1H-pyrazol-4-yl)-1H-thieno[3,2-c]pyrazol-3-yl]-1H-indole-6-sulfonic acid phenylamide |
| 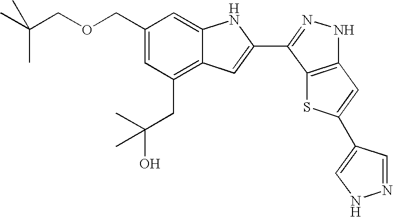 | 1-{6-(2,2-Dimethyl-propoxymethyl)-2-[5-(1H-pyrazol-4-yl)-1H-thieno[3,2-c]pyrazol-3-yl]-1H-indol-4-yl}-2-methyl-propan-2-ol |
| 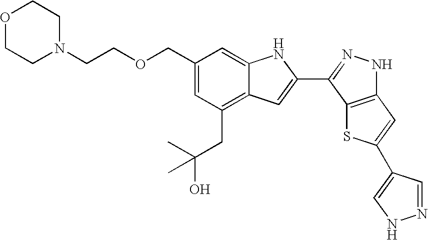 | 2-Methyl-1-{6-(2-morpholin-4-yl-ethoxymethyl)-2-[5-(1H-pyrazol-4-yl)-1H-thieno[3,2-c]pyrazol-3-yl]-1H-indol-4-yl}-propan-2-ol |
| 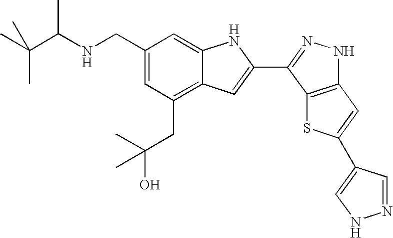 | 2-Methyl-1-{2-[5-(1H-pyrazol-4-yl)-1H-thieno[3,2-c]pyrazol-3-yl]-6-[(1,2,2-trimethyl-propylamino)-methyl]-1H-indol-4-yl}-propan-2-ol |
| 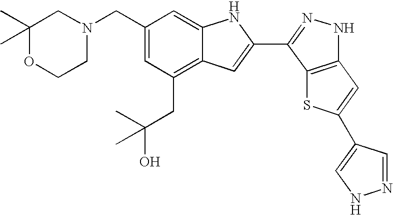 | 1-{6-(2,2-Dimethyl-morpholin-4-ylmethyl)-2-[5-(1H-pyrazol-4-yl)-1H-thieno[3,2-c]pyrazol-3-yl]-1H-indol-4-yl}-2-methyl-propan-2-ol |
| 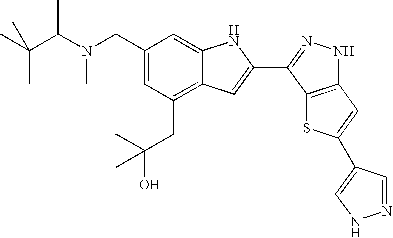 | 2-Methyl-1-{6-{[methyl-(1,2,2-trimethyl-propyl)-amino]-methyl}-2-[5-(1H-pyrazol-4-yl)-1H-thieno[3,2-c]pyrazol-3-yl]-1H-indol-4-yl}-propan-2-ol |
| 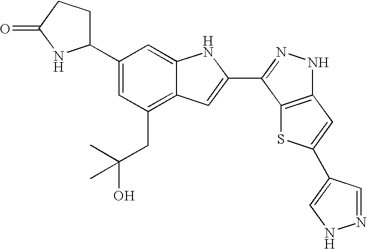 | 5-{4-(2-Hydroxy-2-methyl-propyl)-2-[5-(1H-pyrazol-4-yl)-1H-thieno[3,2-c]pyrazol-3-yl]-1H-indol-6-yl}-pyrrolidin-2-one |
| 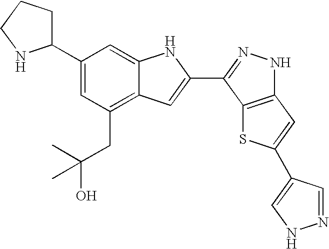 | 2-Methyl-1-{2-[5-(1H-pyrazol-4-yl)-1H-thieno[3,2-c]pyrazol-3-yl]-6-pyrrolidin-2-yl-1H-indol-4-yl}-propan-2-ol |
| 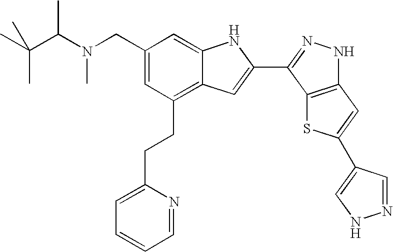 | Methyl-[2-[5-(1H-pyrazol-4-yl)-1H-thieno[3,2-c]pyrazol-3-yl]-4-(2-pyridin-2-yl-ethyl)-1H-indol-6-ylmethyl]-(1,2,2-trimethyl-propyl)-amine |
| 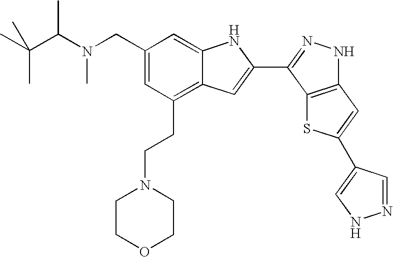 | Methyl-{4-(2-morpholin-4-yl-ethyl)-2-[5-(1H-pyrazol-4-yl)-1H-thieno[3,2-c]pyrazol-3-yl]-1H-indol-6-ylmethyl}-(1,2,2-trimethyl-propyl)-amine |
| 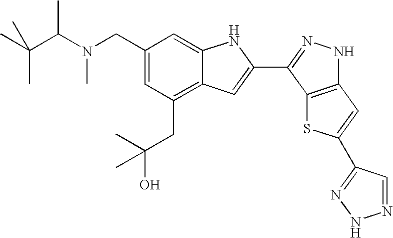 | 2-Methyl-1-{6-{[methyl-(1,2,2-trimethyl-propyl)-amino]-methyl}-2-[5-(2H-[1,2,3]triazol-4-yl)-1H-thieno[3,2-c]pyrazol-3-yl]-1H-indol-4-yl}-propan-2-ol |
| 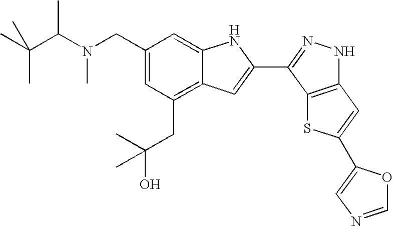 | 2-Methyl-1-[6-{[methyl-(1,2,2-trimethyl-propyl)-amino]-methyl}-2-(5-oxazol-5-yl-1H-thieno[3,2-c]pyrazol-3-yl)-1H-indol-4-yl]-propan-2-ol |
| 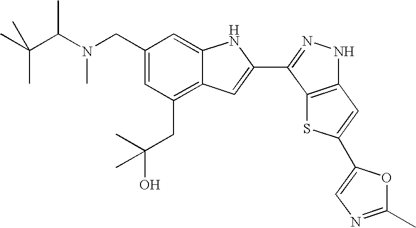 | 2-Methyl-1-(2-[5-(2-methyl-oxazol-5-yl)-1H-thieno[3,2-c]pyrazol-3-yl]-6-{[methyl-(1,2,2-trimethyl-propyl)-amino]-methyl}-1H-indol-4-yl)-propan-2-ol |
| 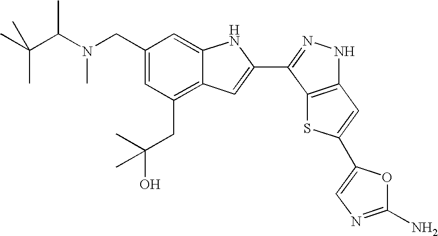 | 1-(2-[5-(2-Amino-oxazol-5-yl)-1H-thieno[3,2-c]pyrazol-3-yl]-6-{[methyl-(1,2,2-trimethyl-propyl)-amino]-methyl}-1H-indol-4-yl)-2-methyl-propan-2-ol |
| 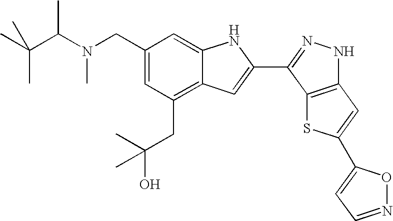 | 1-(2-(5-Isoxazol-5-yl-1H-thieno[3,2-c]pyrazol-3-yl)-6-{[methyl-(1,2,2-trimethyl-propyl)-amino]-methyl}-1H-indol-4-yl)-2-methyl-propan-2-ol |
| 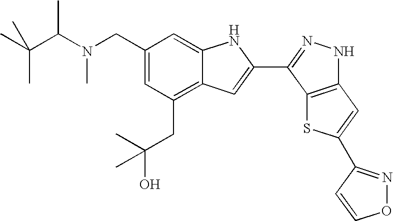 | 1-(2-(5-Isoxazol-3-yl-1H-thieno[3,2-c]pyrazol-3-yl)-6-{[methyl-(1,2,2-trimethyl-propyl)-amino]-methyl}-1H-indol-4-yl)-2-methyl-propan-2-ol |
| 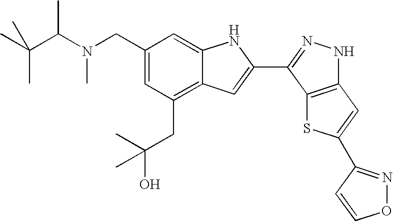 | 1-(2-(5-Isoxazol-3-yl-1H-thieno[3,2-c]pyrazol-3-yl)-6-{[methyl-(1,2,2-trimethyl-propyl)-amino]-methyl}-1H-indol-4-yl)-2-methyl-propan-2-ol |
| 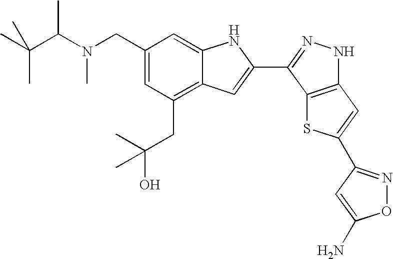 | 1-(2-[5-(5-Amino-isoxazol-3-yl)-1H-thieno[3,2-c]pyrazol-3-yl]-6-{[methyl-(1,2,2-trimethyl-propyl)-amino]-methyl}-1H-indol-4-yl)-2-methyl-propan-2-ol |
| 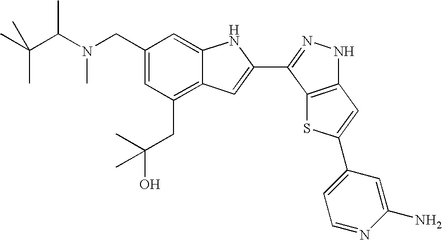 | 1-(2-[5-(2-Amino-pyridin-4-yl)-1H-thieno[3,2-c]pyrazol-3-yl]-6-{[methyl-(1,2,2-trimethyl-propyl)-amino]-methyl}-1H-indol-4-yl)-2-methyl-propan-2-ol |
| 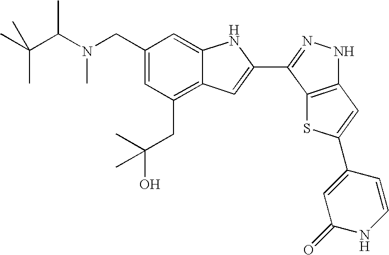 | 4-[3-(4-(2-Hydroxy-2-methyl-propyl)-6-{[methyl-(1,2,2-trimethyl-propyl)-amino]-methyl}-1H-indol-2-yl)-1H-thieno[3,2-c]pyrazol-5-yl]-1H-pyridin-2-one |
| 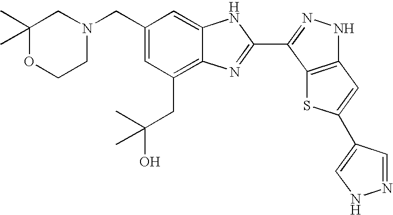 | 1-{6-(2,2-Dimethyl-morpholin-4-ylmethyl)-2-[5-(1H-pyrazol-4-yl)-1H-thieno[3,2-c]pyrazol-3-yl]-1H-benzoimidazol-4-yl}-2-methyl-propan-2-ol |
| 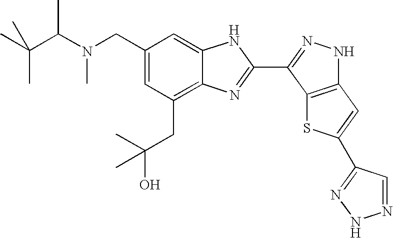 | 2-Methyl-1-{6-{[methyl-(1,2,2-trimethyl-propyl)-amino]-methyl}-2-[5-(2H-[1,2,3]triazol-4-yl)-1H-thieno[3,2-c]pyrazol-3-yl]-1H-benzoimidazol-4-yl}-propan-2-ol |
| 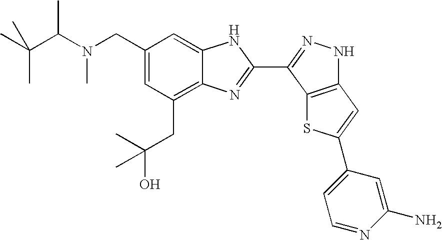 | 1-(2-[5-(2-Amino-pyridin-4-yl)-1H-thieno[3,2-c]pyrazol-3-yl]-6-{[methyl-(1,2,2-trimethyl-propyl)-amino]-methyl}-1H-benzoimidazol-4-yl)-2-methyl-propan-2-ol |

In all the compounds disclosed herein above in this application, in the event the nomenclature is in conflict with the structure, it shall be understood that the compound is defined by the structure.

The invention includes the use of any compounds described above containing one or more asymmetric carbon atoms which may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Of particular importance according to the invention are compounds of formula (I), for use as pharmaceutical compositions with an anti-Tec kinase activity.

The invention also relates to the use of a compound of formula (I), for preparing a pharmaceutical composition for the treatment and/or prevention of a Tec kinase mediated disease or condition.

The invention also relates to pharmaceutical preparations, containing as active substance one or more compounds of formula (I), or the pharmaceutically acceptable derivatives thereof, optionally combined with conventional excipients and/or carriers.

Compounds of the invention also include their isotopically-labelled forms. An isotopically-labelled form of an active agent of a combination of the present invention is identical to said active agent but for the fact that one or more atoms of said active agent have been replaced by an atom or atoms having an atomic mass or mass number different from the atomic mass or mass number of said atom which is usually found in nature. Examples of isotopes which are readily available commercially and which can be incorporated into an active agent of a combination of the present invention in accordance with well established procedures, include isotopes of hydrogen, carbon, nitrogen, oxygen, phosphorous, fluorine and chlorine, e.g., ²H, ³H, ¹³C, ¹⁴C, ¹⁵N, ¹⁸O, ¹⁷O, ³¹P, ³²P, ³⁵S, ¹⁸F., and ³⁶Cl, respectively. An active agent of a combination of the present invention, a prodrug thereof, or a pharmaceutically acceptable salt of either which contains one or more of the above-mentioned isotopes and/or other isotopes of other atoms is contemplated to be within the scope of the present invention.

Some of the compounds of formula (I) can exist in more than one tautomeric form. The invention includes methods using all such tautomers.

All terms as used herein in this specification, unless otherwise stated, shall be understood in their ordinary meaning as known in the art.

Alkyl, alkenyl, alkynyl, alkoxy, alkylthio, acyl, alkoxycarbonyl, acyloxy, acylamino, alkylsulfonyl and all other alkyl containing groups shall be understood unless otherwise specified as being C1-10, branched or unbranched where structurally possible, and optionally partially or fully halogenated. For ‘Cₒ₋ₙ alkyl’, where n is an integer 1,2,3 etc, shall be understood to be a bond when the definition is ‘C₀’, and alkyl when n is greater than or equal to 1. Other more specific definitions are as follows:

BOC or t-BOC is tertiary-butoxycarbonyl.

t-Bu is tertiary-butyl.

DMF is dimethylformamide.

EtOAc is ethyl acetate.

EtOH and MeOH are ethanol and methanol, respectively.

TFA is trifluoroacetic acid.

THF is tetrahydrofuran.

DMSO is dimethylsulfoxide.

TBTU is O-(1H-benzotriazol-1-yl)-N,N,N′,N′-tetramethyluronium tetrafluoroborate.

FMOC is 9-fluorenylmethoxycarbonyl.

The term “aroyl” as used in the present specification shall be understood to mean “benzoyl” or “naphthoyl”.

The term “carbocycle” shall be understood to mean an aliphatic hydrocarbon radical containing from three to twelve carbon atoms. Carbocycles include hydrocarbon rings containing from three to ten carbon atoms. These carbocycles may be either aromatic and non-aromatic ring systems, and optionally or fully halogenated. The non-aromatic ring systems may be mono- or polyunsaturated. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms selected from nitrogen, oxygen and sulfur. The heterocycle may be attached by any atom of the cycle, which results in the creation of a stable structure. Unless otherwise stated, heterocycles include but are not limited to, pyrrolidinyl, morpholinyl, thiomorpholinyl, dioxalanyl, piperidinyl, piperazinyl, aziridinyl and tetrahydrofuranyl.

The term “heteroaryl” shall be understood to mean an aromatic 5-8 membered monocyclic or 8-11 membered bicyclic ring containing 1-4 heteroatoms such as N,O and S. Unless otherwise stated, such heteroaryls include but are not limited to thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl and indazolyl.

The term “heteroatom” as used herein shall be understood to mean atoms other than carbon such as O, N, S and P.

In all alkyl groups or carbon chains within cycloalkyl groups, where one or more carbon atoms are optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain.

Substitution on a carbon such as a methylene carbon by groups such as oxo result in definitions such as: alkoxycarbonyl, acyl, and amido, or if substituted on a ring can, for example, replace a methylene group —CH₂— with a carbonyl >C═O.

The term “aryl” as used herein shall be understood to mean aromatic carbocycle or heteroaryl as defined herein. Each aryl or heteroaryl unless otherwise specified includes its partially or fully hydrogenated derivative. For example, quinolinyl may include decahydroquinolinyl and tetrahydroquinolinyl, naphthyl may include its hydrogenated derivatives such as tetrahydranaphthyl. Each may be partially or fully halogenated. Other partially or fully hydrogenated derivatives of the aryl and heteroaryl compounds described herein will be apparent to one of ordinary skill in the art.

Terms which are analogs of the above cyclic moieties such as aryloxy or heteroaryl amine shall be understood to mean an aryl, heteroaryl, heterocycle as defined above attached to it's respective functional group.

As used herein, “nitrogen” and “sulfur” include any oxidized form of nitrogen and sulfur and the quaternized form of any basic nitrogen. For example, for an alkylthio radical such as —S—C₁₁ alkyl, unless otherwise specified, this shall be understood to include —S(O)—C₁₋₆ alkyl and —S(O)₂—C₁₋₆ alkyl.

The term “halogen” as used in the present specification shall be understood to mean bromine, chlorine, fluorine or iodine. The definitions “partially or fully halogenated” “substituted by one or more halogen atoms” includes for example, mono, di or tri halo derivatives on one or more carbon atoms. A non-limiting example would be a halogenated alkyl such as —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The term “patient” refers to a warm-blooded mammal and preferably, a human.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formula (I).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the formula (I). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed herein above, thereby imparting the desired pharmacological effect.

### Methods of Therapeutic Use

The compounds of the invention are effective inhibitors of Tec kinase family activity, especially of Itk. Therefore, in one embodiment of the invention, there is provided methods of treating immunological disorders using compounds of the invention. In another embodiment, there is provided methods of treating inflammatory disorders using compounds of the invention. In yet another embodiment, there is provided methods of treating allergic disorders using compounds of the invention. In yet still another embodiment, there is provided methods of enhancing memory cell generation for vaccines using compounds of the invention. In a further embodiment, there is provided methods of treating cell proliferative disorders using compounds of the invention.

Without wishing to be bound by theory, the compounds of this invention modulate T cell and mast cell activation via effective inhibition of Itk. The inhibition of T cell activation is therapeutically useful for selectively suppressing immune function. Thus, the inhibition of Itk is an attractive means for preventing and treating a variety of immune disorders, including inflammatory diseases, autoimmune diseases, organ and bone marrow transplant rejection and other disorders associated with T cell mediated immune response. In particular, the compounds of the invention may be used to prevent or treat acute or chronic inflammation, allergies, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease (and other forms of organ or bone marrow transplant rejection) and lupus erythematosus.

The compounds of the invention are also effective inhibitors of Tec family kinases other than Itk including Txk, Tec, Btk, and Bmx and would thus be useful in treating diseases associated with the activity of one or more of these Tec family kinases.

Inhibitors of mast cell activation and degranulation block the release of allergic and pro-inflammatory mediators and cytokines. Thus inhibitors of ltk have potential utility in treating inflammatory and allergic disorders, including asthma, chronic obstructive pulmonary disease (COPD), adult respiratory distress syndrome (ARDS), bronchitis, conjunctivitis, dermatitis and allergic rhinitis. Other disorders associated with T cell or mast cell mediated immune response will be evident to those of ordinary skill in the art and can also be treated with the compounds and compositions of this invention. Inhibitors of Itk and other Tec family kinases have potential utility in combination with other therapies for the treatment of immune, inflammatory, proliferative, and allergic disorders. Examples, though not all encompassing, include co-administration with steroids, leukotriene antagonists, anti-histamines, cyclosporin, or rapamycin.

One strategy to improve vaccination methods is to increase the number of memory T cells generated. As described in the Background, in the absence of Itk in mice, increased numbers of memory cells are generated. Thus, within the scope of the invention is the use of the present compounds in the formulation of improved vaccines that generate increased numbers of memory T cells.

For therapeutic use, the compounds of the invention may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds of this invention may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the compounds of this invention include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

### Biological Activity

### Tec Family Kinase Assay

Itk, Txk, Tec, Btk, and Bmx are purified as a GST-fusion protein. The kinase activity is measured using DELFIA (Dissociation Enhanced Lanthanide Fluoroimmunoassay) which utilizes europium chelate-labeled anti-phosphotyrosine antibodies to detect phosphate transfer to a random polymer, poly Glu₄: Tyr₁ (PGTYR). The screen is run on the Zymark Allegro robot system to dispense reagents, buffers and samples for assay, and also to wash and read plates. The kinase assay is performed in kinase assay buffer (50 mM HEPES, pH 7.0, 25 mM MgCl₂, 5 mM MnCl₂, 50 mM KCl, 100 μM Na₃VO₄, 0.2% BSA, 0.01% CHAPS, 200 μM TCEP). Test samples initially dissolved in DMSO at 1 mg/mL, are pre-diluted for dose response (10 doses with starting final concentration of 3 μg/mL, 1 to 3 serial dilutions) with the assay buffer in 96-well polypropylene microtiter plates. A 50 μL volume/well of a mixture of substrates containing ATP (final ATP concentration in each kinase assay is equal to its apparent ATP Kₘ) and 3.6 ng/μL PGTYR-biotin (CIS Bio International) in kinase buffer is added to neutravidin coated 96-well white plate (PIERCE), followed by 25 mL/well test sample solution and 25 μL/well of diluted enzyme (1-7 nM final conc.). Background wells are incubated with buffer, rather than 25 μL enzyme. The assay plates are incubated for 30 min at room temperature. Following incubation, the assay plates are washed three times with 250 μL DELFIA wash buffer. A 100 μL aliquot of 1 nM europium-labeled anti-phosphotyrosine (Eu³⁺-PT66, Wallac CR04-100) diluted in DELFIA assay buffer is added to each well and incubated for 30 min at room temperature. Upon completion of the incubation, the plate is washed four times with 250 μL of wash buffer and 100 μL of DELFIA Enhancement Solution (Wallac) is added to each well. After 15 min of longer, time-resolved fluorescence is measured (excitation at 360 nm, emission at 620 nm) after a delay time of 250 μs.

Preferred compounds of the invention have an activity of 1 microMolar or less.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the schemes below are either commercially available or easily prepared from commercially available materials by those skilled in the art.

### General Synthetic Methods

The invention also provides processes for making compounds of formula (I). Intermediates used in the preparation of compounds of the invention are either commercially available or readily prepared by methods known to those skilled in the art. Further reference in this regard may be made to WO 05/026175, WO 03/041708 corresponding to US publication US 2003-0144281, and PCT application PCT/US03/24024 corresponding to US publication 2005-0203158.

Compounds of formula (I) may be prepared as illustrated in Scheme 1. An optionally substituted 3-iodo-thieno[3,2-c]pyrazole (II), with the 1-position protected with a suitable protecting group such as the t-Boc group shown, may be arylated by reaction with an optionally substituted indole-2-boronic acid (III, X═C) or benzimidazole-2-boronic acid (III, X═N)) utilizing palladium catalyzed cross coupling reaction chemistry well known in the art (see for example, A. Suzuki, J. Organomet. Chem. 1999, 576, 147) to provide intermediate (IV), where R1, R2 or AR shall have the meaning given for the formula (I) of the invention described herein above. Intermediate IV may be converted to the desired compound of formula (I) utilizing deprotecting chemistry well known in the art (see for example, ‘Protective Groups in Organic Synthesis’, 3^{rd} edition, T. W. Greene & P. G. M. Wuts, Wiley-Interscience (1999)).

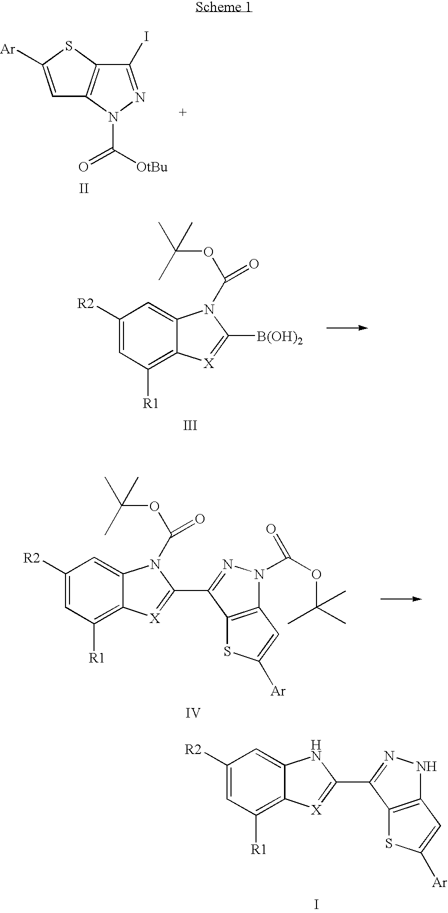

### SYNTHETIC EXAMPLES

### Example 1

### 3-Iodo-thieno[3,2-c]pyrazole-1,5-dicarboxylic acid 1-tert-butyl ester 5-methyl ester

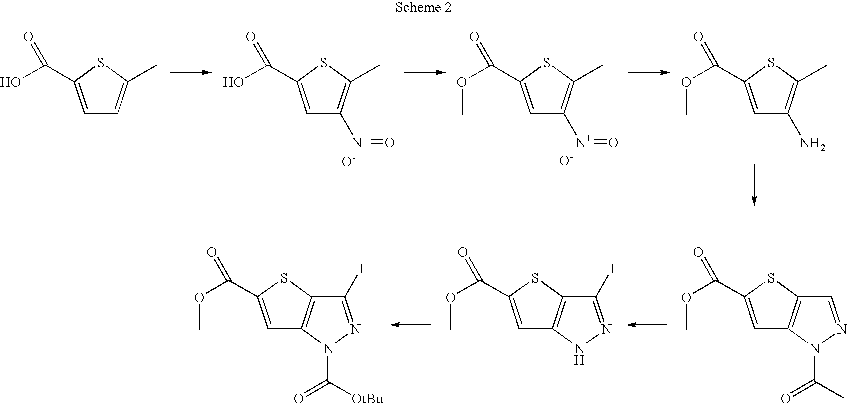

To a solution of acetic anhydride (70 mL) on ice was added 25 mL of fuming nitric acid (>90%) slowly. This solution was stirred and cooled to −10° C. To this stirred solution was added 16g of 5-methylthiophene-2-carboxylic acid in small portions. The solution turned dark orange over time and gas was evolved. The reaction was allowed to warm to room temperature overnight, then cool to −20° C. and the resulting light orange solidwas collected by filtration. The solid was washed with 100 mL of water and dried to provide 7.6 g (36%) of 5-methyl-4-nitro-thiophene-2-carboxylic acid as a yellow solid.

To a solution of 5-methyl-4-nitrothiophene 2-carboxylic acid (7.6 g) in 150 mL of methanol was added 5 mL concentrated sulfuric acid. After stirring for 16 h, the mixture was cooled on ice and neutralized with saturated sodium bicarbonate solution. The product was extracted with ethyl acetate (1×50 ml). The separated organic layer was dried, filtered and concentrated, and passed through a silica gel plug, eluting with 50% ethyl acetate/hexanes. The filtrate was concentrated to give 5.4 g (66%) of 5-methyl-4-nitrothiophene 2-carboxylic acid methyl ester as a solid.

### 1-Acetyl-1H-thieno[3,2-c]pyrazole-5-carboxylic acid methyl ester

A mixture of 5-methyl-4-nitrothiophene 2-carboxylic acid methyl ester (2.0 g) in 20 ML of methanol and 1 mL of a slurry of 50% Raney nickel in water was shaken under 40 psi of hydrogen gas for 16 h. The reaction mixture was filtered through diatomaceous earth and the resulting aminothiophene was taken on without further purification.

To a solution of aminothiophene (336 mg, 1.96 mmol) in 10 mL of toluene was added potassium acetate (116 mg, 1.18 mmol) and acetic anhydride (3.15 mL, 33.4 mmol). The solution was heated to 93° C. To this solution was added isoamyl nitrite (0.264 mL, 1.96 mmol) and the solution was stirred at 93° C. for 12 hours. The solution was cooled to room temperature and diluted with 25 mL of ethyl acetate, and washed with saturated sodium bicarbonate solution and saturated sodium chloride solution. The organics were dried, filtered and concentrated and purified by flash chromatography eluting with a gradient of 0-100% hexanes/ethyl acetate to yield 240 mg (55%) of the title compound.

To a solution of 1-acetyl-1H-thieno[3,2-c]pyrazole-5-carboxylic acid methyl ester (217 mg, 0.969 mmol) in 2 mL of methanol was added 5 mL of a 25% wt solution of sodium methoxide in methanol. The solution was stirred at 60° C. for 20 minutes, and then cooled to room temperature. A solution of iodine (295 mg, 1.16 mmol) in 2 mL of N,N-dimethylformamide was added and the solution was heated to 60° C. for 1 hour. The solution was cooled and concentrated, then diluted with ethyl acetate and water. The aqueous layer was separated and back extracted (2×10 mL) with ethyl acetate. The combined organic layers were dried over magnesium sulfate, filtered and concentrated. Some starting material was observed, so the compound was resubmitted to the above reaction conditions and allowed to stir at 60° C. for 48 hours following iodine/N,N-dimethylformamide treatment. The solution was again concentrated, diluted with ethyl acetate and water, and the aqueous layer was back extracted with ethyl acetate (2×10 mL). The combined organics were dried over magnesium sulfate, filtered and concentrated. The crude product was purified by flash chromatography eluting with a gradient of 10%-100% hexanes/ethyl acetate to yield 221 mg (74%) of 3-iodo-1H-thieno[3,2-c]pyrazole-5-carboxylic acid methyl ester.

To a solution of 3-iodo-1H-thieno[3,2-c]pyrazole-5-carboxylic acid methyl ester (221 mg, 0.715 mmol) in 5 mL of THF was added 172 mg (0.787 mmol) of di-t-butyl dicarbonate and a catalytic amount of 4-dimethylaminopyridine. The solution was stirred for 14 hours and then diluted with water and extracted with ethyl acetate (3×10 ml). The combined organic layers were dried over magnesium sulfate, filtered and concentrated to give 280 mg (96%) of the title compound.

### Example 2

### 4-(3-Hydroxy-3-methyl-butyl)-6-{[methyl-(1,2,2-trimethyl-propyl)-amino]-methyl}-indole-1-carboxylic acid tert-butyl ester

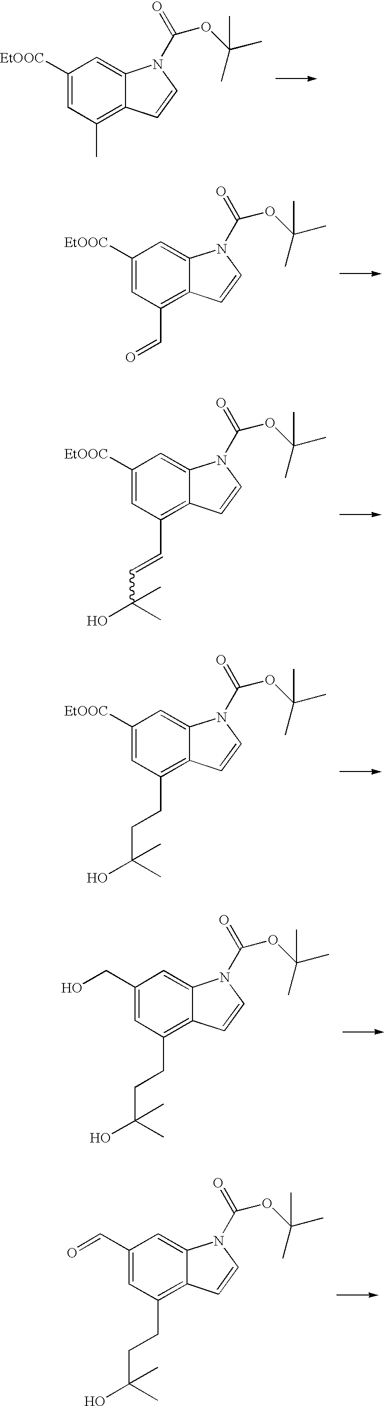

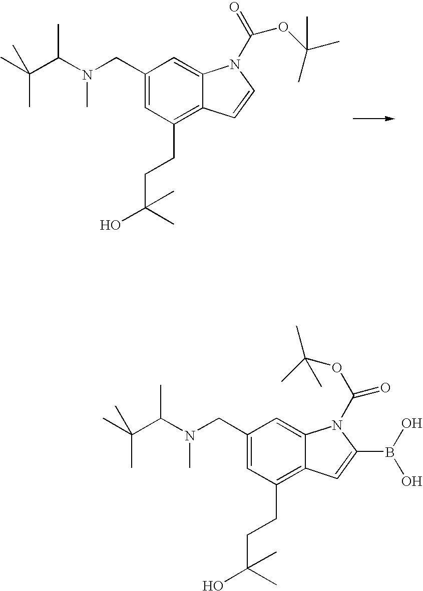

A solution of 606 mg (2.0 mmol) of 4-methyl-indole-1,6-dicarboxylic acid 1-tert-butyl ester 6-ethyl ester in 25 mL of dichloromethane is treated with 1.0 g (6.3 mmol) of finely ground potassium permanganate and 3.0 g (34.5 mmol) of manganese dioxide at room temperature. The resulting mixture is stirred at this temperature for 16 h and filtered through diatomaceous earth. The filtrate is washed with water and brine, dried with magnesium sulfate, filtered and concentrated. The residue is purified by flash chromatography to give 4-formyl-indole-1,6-dicarboxylic acid 1-tert-butyl ester 6-ethyl ester.

A solution of 612 mg (4.0 mol) of 1-bromo-2-methyl-propan-2-ol and 1.05 g (4.0 mmol) of triphenylphosphine in 30 mL of toluene is refluxed for 16 h and the mixture is cooled to room temperature. Then the mixture is treated with 494 mg (4.4 mmol) of potassium tert-butoxide at room temperature. The resulting mixture is stirred at this temperature for 1 h. To this mixture is added 635 mg (2.0 nmol) of 4-formyl-indole-1,6-dicarboxylic acid 1-tert-butyl ester 6-ethyl ester, and the resulting mixture is stirred at room temperature for 16 h. The mixture is concentrated and the residue is diluted with saturated ammonium chloride solution. The product is extracted with ethyl acetate. The organic layer is washed with water and brine, dried (magnesium sulfate), filtered and concentrated. The residue is purified by flash chromatography to give 4-(3-hydroxy-3-methyl-but-1-enyl)-indole-1,6-dicarboxylic acid 1-tert-butyl ester 6-ethyl ester.

A mixture of 745 mg (2.0 mmol) of 4-(3-hydroxy-3-methyl-but-1-enyl)-indole-1,6-dicarboxylic acid 1-tert-butyl ester 6-ethyl ester and 75 mg of 10 palladium on carbon in 30 mL of methanol is stirred at room temperature under hydrogen gas atmosphere for 4 h. The resulting mixture is filtered through diatomaceous earth, and the filtrate is concentrated to give 4-(3-hydroxy-3-methyl-butyl)-indole-1,6-dicarboxylic acid 1-tert-butyl ester 6-ethyl ester.

A solution of 751 mg (2.0 mmol) of 4-(3-hydroxy-3-methyl-butyl)-indole-1,6-dicarboxylic acid 1-tert-butyl ester 6-ethyl ester in 20 mL of tetrahydrofuran is treated with a solution of 6.0 mL (6.0 mmol) of 1.0 M diisobutylaluminium hydride in tetrahydrofuran at −20° C. The resulting mixture is stirred at this temperature for 6 h and the mixture is quenched with water and sat. ammonium chloride solution. The product is extracted with ethyl acetate. The organic layer is washed with water and brine, dried (magnesium sulfate), filtered and concentrated. The residue is purified by flash chromatography to give 6-hydroxymethyl-4-(3-hydroxy-3-methyl-butyl)-indole-1-carboxylic acid tert-butyl ester

A solution of 667 mg (2.0 mmol) of 6-hydroxymethyl-4-(3-hydroxy-3-methyl-butyl)-indole-1-carboxylic acid tert-butyl ester in 40 mL of dichloromethane is treated with 1.7 g (4.0 mmol) of 1,1,1-tris(acetyloxy)-1,1-dihydro-1,2-benziodoxol-3-(1H)-one (Dess-Martin periodinane) at room temperature. The resulting mixture is stirred at this temperature for 16 h and the diluted with water. The product is extracted with ethyl acetate. The organic layer is washed with water and brine, dried (magnesium sulfate), filtered and concentrated. The residue is purified by flash chromatography to give 6-formyl-4-(3-hydroxy-3-methyl-butyl)-indole-1-carboxylic acid tert-butyl ester.

A solution of 663 mg (2.0 mmol) of 6-formyl-4-(3-hydroxy-3-methyl-butyl)-indole-1-carboxylic acid tert-butyl ester and 2.3 g (20 mmol) of methyl-(1,2,2-trimethyl-propyl)-amine in 50 mL of THF and 50 mL of triethyl orthoformate is treated with 1 mL of acetic acid at room temperature. The resulting mixture is stirred at room temperature for 16 h. To this mixture is added in portions 151 mg (4.0 mmol) of sodium borohydride at room temperature. The resulting mixture is stirred at this temperature for 4 h. The reaction mixture is diluted with saturated sodium bicarbonate solution and the product is extracted with ethyl acetate. The organic layer is washed with brine, dried (sodium sulfate), filtered and concentrated. The residue is purified by flash chromatography to give 4-(3-hydroxy-3-methyl-butyl)-6-{[methyl-(1,2,2-trimethyl-propyl)-amino]-methyl}-indole-1-carboxylic acid tert-butyl ester.

A solution of 861 mg (2.0 mmol) of 4-(3-hydroxy-3-methyl-butyl)-6-{[methyl-(1,2,2-trimethyl-propyl)-amino]-methyl}-indole-1-carboxylic acid tert-butyl ester and 602 mg (3.2 mmol) of triisopropyl borate in 20 mL of THF is treated with 2.4 mL (4.8 mmol) of 2.0 M lithium diisopropylamide solution in heptane/tetrahydrofuran/ethylbenzene at −10° C. The resulting mixture is stirred at this temperature for 2 h and then quenched with 2 N HCl solution. The mixture is stirred at room temperature for 1 h and the pH of this mixture is neutralized with saturated sodium bicarbonate solution. The product is extracted with ethyl acetate. The organic layer is washed with brine, dried (sodium sulfate), filtered and concentrated. The residue is purified by flash chromatography to give the title compound.

### Example 3

### 2-Methyl-4-[6-{[methyl-(1,2,2-trimethyl-propyl)-amino]-methyl}-2-(5-oxazol-5-yl-1H-thieno[3,2-c]pyrazol-3-yl)-1H-indol-4-yl]-butan-2-ol

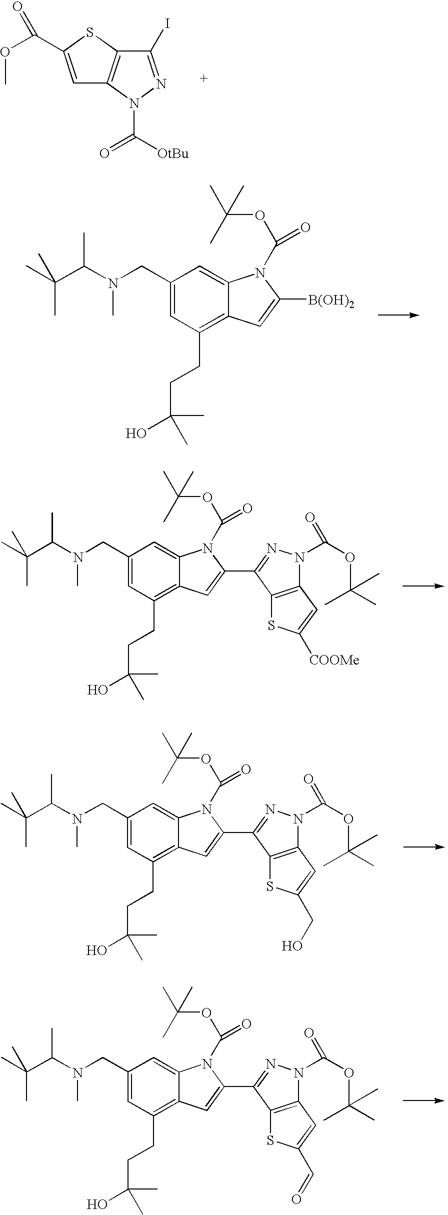

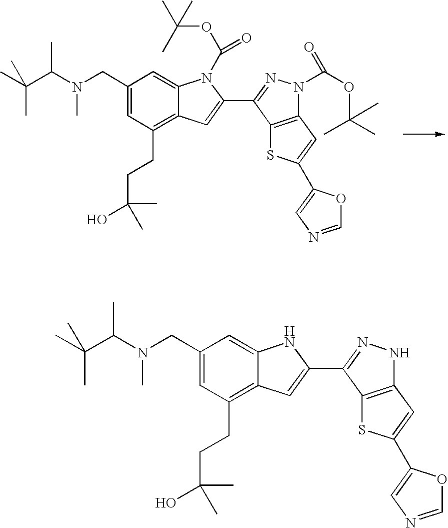

A solution of 816 mg (2.0 mmol) of 3-iodo-thieno[3,2-c]pyrazole-1,5-dicarboxylic acid 1-tert-butyl ester 5-methyl ester, 949 mg (2.0 mmol) of 4-(3-hydroxy-3-methyl-butyl)-6-{[methyl-(1,2,2-trimethyl-propyl)-amino]-methyl}-indole-2-boronic acid-1-carboxylic acid tert-butyl ester and 70 mg (0.1 mmol) of bis(triphenylphosphine)palladium(II) dichloride in 20 mL of dimethyl acetate and 20 mL of 2.0 M potassium carbonate solution in water is heated at 80° C. for 16 hours. The reaction mixture is diluted water and the product is extracted with ethyl acetate. The organic layer is washed with brine, dried (sodium sulfate), filtered and concentrated. The residue is purified by flash chromatography to give 3-(1-tert-butoxycarbonyl-4-(3-hydroxy-3-methyl-butyl)-6-{[methyl-(1,2,2-trimethyl-propyl)-amino]-methyl}-1H-indol-2-yl)-thieno[3,2-c]pyrazole-1,5-dicarboxylic acid 1-tert-butyl ester 5-methyl ester.

A solution of 1422 mg (2.0 mmol) of 3-(1-tert-butoxycarbonyl-4-(3-hydroxy-3-methyl-butyl)-6-{[methyl-(1,2,2-trimethyl-propyl)-amino]-methyl}-1H-indol-2-yl)-thieno[3,2-c]pyrazole-1,5-dicarboxylic acid 1-tert-butyl ester 5-methyl ester in 40 mL of tetrahydrofuran is treated with a solution of 6.0 mL (6.0 mmol) of 1.0 M diisobutylaluminium hydride in tetrahydrofuran at −20° C. The resulting mixture is stirred at this temperature for 6 h and the mixture is quenched with water and saturated ammonium chloride solution. The product is extracted with ethyl acetate. The organic layer is washed with water and brine, dried (magnesium sulfate), filtered and concentrated. The residue is purified by flash chromatography to give 2-(1-tert-butoxycarbonyl-5-hydroxymethyl-1H-thieno[3,2-c]pyrazol-3-yl)-4-(3-hydroxy-3-methyl-butyl)-6-{[methyl-(1,2,2-trimethyl-propyl)-amino]-methyl}-indole-1-carboxylic acid tert-butyl ester.

A solution of 1421 mg (2.0 mmol) of 2-(1-tert-butoxycarbonyl-5-hydroxymethyl-1H-thieno[3,2-c]pyrazol-3-yl)-4-(3-hydroxy-3-methyl-butyl)-6-{[methyl-(1,2,2-trimethyl-propyl)-amino]-methyl}-indole-1-carboxylic acid tert-butyl ester in 50 mL of dichloromethane is treated with 1.7 g (4.0 mmol) of 1,1,1-tris(acetyloxy)-1,1-dihydro-1,2-benziodoxol-3-(1H)-one (Dess-Martin periodinane) at room temperature. The resulting mixture is stirred at this temperature for 16 h and the diluted with water. The product is extracted with ethyl acetate. The organic layer is washed with water and brine, dried (magnesium sulfate), filtered and concentrated. The residue is purified by flash chromatography to give 2-(1-tert-butoxycarbonyl-5-formyl-1H-thieno[3,2-c]pyrazol-3-yl)-4-(3-hydroxy-3-methyl-butyl)-6-{[methyl-(1,2,2-trimethyl-propyl)-amino]-methyl}-indole-1-carboxylic acid tert-butyl ester.

A solution of 1.36 g (2.0 mmol) of 2-(1-tert-butoxycarbonyl-5-formyl-1H-thieno[3,2-c]pyrazol-3-yl)-4-(3-hydroxy-3-methyl-butyl)-6-{[methyl-(1,2,2-trimethyl-propyl)-amino]-methyl}-indole-1-carboxylic acid tert-butyl ester, 395 g (2.0 mmol) of tosylmethyl isocyanide and 276 mg (2.0 mmol) of potassium carbonate in 50 mL of MeOH is refluxed for 0.5 hour. The solvent is evaporated under reduced pressure. The resulting residue is poured into ice-water, and extracted with ethyl acetate. The extract is washed with saturated ammonium chloride solution, water, brine and dried over magnesium sulfate. The organic solvent is evaporated under reduced pressure and the residue is purified by flash chromatography to give 2-(1-tert-butoxycarbonyl-5-oxazol-5-yl-1H-thieno[3,2-c]pyrazol-3-yl)-4-(3-hydroxy-3-methyl-butyl)-6-{[methyl-(1,2,2-trimethyl-propyl)-amino]-methyl}-indole-1-carboxylic acid tert-butyl ester

A solution of 1.44 g (2.0 mmol) of 2-(1-tert-butoxycarbonyl-5-oxazol-5-yl-1H-thieno[3,2-c]pyrazol-3-yl)-4-(3-hydroxy-3-methyl-butyl)-6-{[methyl-(1,2,2-trimethyl-propyl)-amino]-methyl}-indole-1-carboxylic acid tert-butyl ester in 30 mL of dichloromethane and 30 mL of trifluoroacetic acid is stirred at room temperature for 16 h. The reaction mixture is concentrated and diluted with water. The pH of this solution is neutralized with 1 N sodium hydroxide solution. The product is extracted with ethyl acetate. The organic layer is washed with water and brine, dried (magnesium sulfate), filtered and concentrated. The residue is purified flash chromatography to give the title compound.

1. A compound of the formula (I): 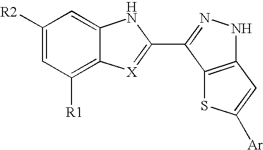
wherein:
X is CH or N;
Ar is chosen from 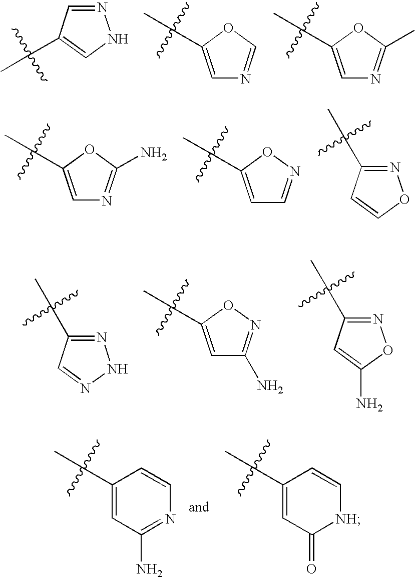
R1 is 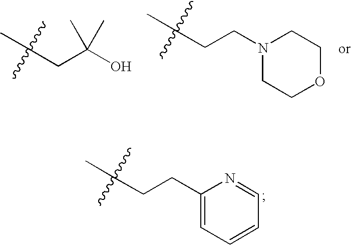
R2 is 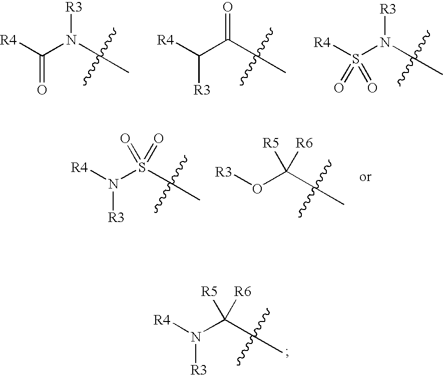
R3, R4 are independently chosen from hydrogen, alkyl, carbocycle, heterocycle C0-5alkyl and heteroaryl wherein each cycloalkyl, heterocycle, aryl and heteroaryl are optionally substituted by C1-5alkyl;
or R3 and R4 optionally combine together to form following rings: 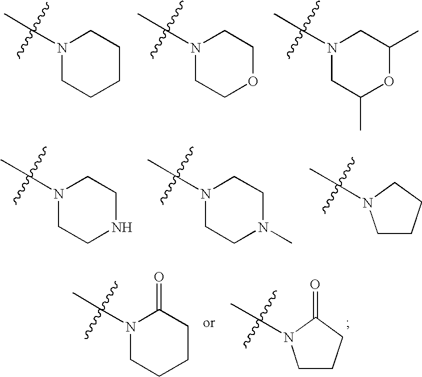
R5, R6 are independently chosen from hydrogen, C1-5alkyl, or optionally combine together to form 3, 4, 5 or 6 membered cycloalkyl ring;
or the pharmaceutically acceptable salts thereof.

2. The compound according to claim 1 and wherein:
X is CH;
R3, R4 are independently chosen from hydrogen, C1-5 alkyl, C4-8cycloalkyl, phenyl, naphthyl, morpholinyl, morpholinyl C1-5alkyl, pyrrolidine, pyrrolidinone, benzothiophenyl, benzodioxolyl, quinolinyl, indolyl, thiazolyl, thienyl, furanyl, isoxazolyl, oxazolyl, imidazolyl, thiadiazolyl, pyrazolyl, pyrazinyl and pyridinyl wherein each cycloalkyl, heterocycle, aryl and heteroaryl are optionally substituted by C1-5alkyl; or R3 and R4 optionally combine together to form following rings:

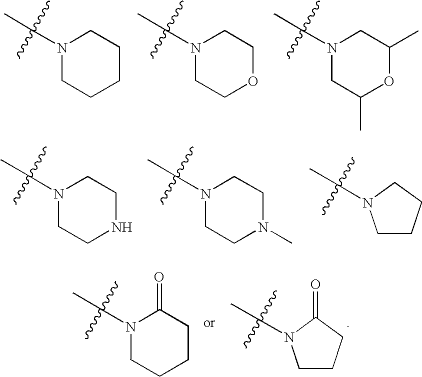

3. The compound according to claim 2 and wherein:
R3, R4 are independently chosen from hydrogen, C₁₋₅ alkyl, C₄₋₆cycloalkyl, phenyl, naphthyl, morpholinyl, morpholinyl, morpholinyl C1-5alkyl, pyrrolidine, pyrrolidinone, wherein each cycloalkyl, heterocycle and aryl are optionally substituted by C1-5alkyl; or R3 and R4 optionally combine together to form following rings:

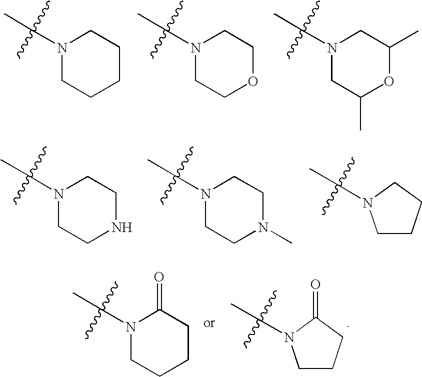

4. A compound wherein the compound is chosen from
N-{4-(2-Hydroxy-2-methyl-propyl)-2-[5-(1H-pyrazol-4-yl)-1H-thieno[3,2-c]pyrazol-3-yl]-1H-indol-6-yl}-N-methyl-benzamide
Pyridine-2-carboxylic acid {4-(2-hydroxy-2-methyl-propyl)-2-[5-(1H-pyrazol-4-yl)-1H-thieno[3,2-c]pyrazol-3-yl]-1H-indol-6-yl}-methyl-amide
Cyclohexanecarboxylic acid {4-(2-hydroxy-2-methyl-propyl)-2-[5-(1H-pyrazol-4-yl)-1H-thieno[3,2-c]pyrazol-3-yl]-1H-indol-6-yl}-methyl-amide
4-(2-Hydroxy-2-methyl-propyl)-2-[5-(1H-pyrazol-4-yl)-1H-thieno[3,2-c]pyrazol-3-yl]-1H-indole-6-carboxylic acid methyl-phenyl-amide
4-(2-Hydroxy-2-methyl-propyl)-2-[5-(1H-pyrazol-4-yl)-1H-thieno[3,2-c]pyrazol-3-yl]-1H-indole-6-carboxylic acid cyclohexyl-methyl-amide
N-{4-(2-Hydroxy-2-methyl-propyl)-2-[5-(1H-pyrazol-4-yl)-1H-thieno[3,2-c]pyrazol-3-yl]-1H-indol-6-yl}-N-methyl-benzenesulfonamide
4-(2-Hydroxy-2-methyl-propyl)-2-[5-(1H-pyrazol-4-yl)-1H-thieno[3,2-c]pyrazol-3-yl]-1H-indole-6-sulfonic acid phenylamide
1-{6-(2,2-Dimethyl-propoxymethyl)-2-[5-(1H-pyrazol-4-yl)-1H-thieno[3,2-c]pyrazol-3-yl]-1H-indol-4-yl}-2-methyl-propan-2-ol
2-Methyl-1-{6-(2-morpholin-4-yl-ethoxymethyl)-2-[5-(1H-pyrazol-4-yl)-1H-thieno[3,2-c]pyrazol-3-yl]-1H-indol-4-yl}-propan-2-ol
2-Methyl-1-{2-[5-(1H-pyrazol-4-yl)-1H-thieno[3,2-c]pyrazol-3-yl]-6-[(1,2,2-trimethyl-propylamino)-methyl]-1H-indol-4-yl}-propan-2-ol
1-{6-(2,2-Dimethyl-morpholin-4-ylmethyl)-2-[5-(1H-pyrazol-4-yl)-1H-thieno[3,2-c]pyrazol-3-yl]-1H-indol-4-yl}-2-methyl-propan-2-ol
2-Methyl-1-{6-{[methyl-(1,2,2-trimethyl-propyl)-amino]-methyl}-2-[5-(1H-pyrazol-4-yl)-1H-thieno[3,2-c]pyrazol-3-yl]-1H-indol-4-yl}-propan-2-ol
5-{4-(2-Hydroxy-2-methyl-propyl)-2-[5-(1H-pyrazol-4-yl)-1H-thieno[3,2-c]pyrazol-3-yl]-1H-indol-6-yl}-pyrrolidin-2-one
2-Methyl-1-{2-[5-(1H-pyrazol-4-yl)-1H-thieno[3,2-c]pyrazol-3-yl]-6-pyrrolidin-2-yl-1H-indol-4-yl}-propan-2-ol
Methyl-[2-[5-(1H-pyrazol-4-yl)-1H-thieno[3,2-c]pyrazol-3-yl]-4-(2-pyridin-2-yl-ethyl)-1H-indol-6-ylmethyl]-(1,2,2-trimethyl-propyl)-amine
Methyl-{4-(2-morpholin-4-yl-ethyl)-2-[5-(1H-pyrazol-4-yl)-1H-thieno[3,2-c]pyrazol-3-yl]-1H-indol-6-ylmethyl}-(1,2,2-trimethyl-propyl)-amine
2-Methyl-1-{6-{[methyl-(1,2,2-trimethyl-propyl)-amino]-methyl}-2-[5-(2H-[1,2,3]triazol-4-yl)-1H-thieno[3,2-c]pyrazol-3-yl]-1H-indol-4-yl}-propan-2-ol
2-Methyl-1-[6-{[methyl-(1,2,2-trimethyl-propyl)-amino]-methyl}-2-(5-oxazol-5-yl-1H-thieno[3,2-c]pyrazol-3-yl)-1H-indol-4-yl]-propan-2-ol
2-Methyl-1-(2-[5-(2-methyl-oxazol-5-yl)-1H-thieno[3,2-c]pyrazol-3-yl]-6-{[methyl-(1,2,2-trimethyl-propyl)-amino]-methyl}-1H-indol-4-yl)-propan-2-ol
1-(2-[5-(2-Amino-oxazol-5-yl)-1H-thieno[3,2-c]pyrazol-3-yl]-6-{[methyl-(1,2,2-trimethyl-propyl)-amino]-methyl}-1H-indol-4-yl)-2-methyl-propan-2-ol
1-(2-(5-Isoxazol-5-yl-1H-thieno[3,2-c]pyrazol-3-yl)-6-{[methyl-(1,2,2-trimethyl-propyl)-amino]-methyl}-1H-indol-4-yl)-2-methyl-propan-2-ol
1-(2-(5-Isoxazol-3-yl-1H-thieno[3,2-c]pyrazol-3-yl)-6-{[methyl-(1,2,2-trimethyl-propyl)-amino]-methyl}-1H-indol-4-yl)-2-methyl-propan-2-ol
1-(2-(5-Isoxazol-3-yl-1H-thieno[3,2-c]pyrazol-3-yl)-6-{[methyl-(1,2,2-trimethyl-propyl)-amino]-methyl}-1H-indol-4-yl)-2-methyl-propan-2-ol
1-(2-[5-(5-Amino-isoxazol-3-yl)-1H-thieno[3,2-c]pyrazol-3-yl]-6-{[methyl-(1,2,2-trimethyl-propyl)-amino]-methyl}-1H-indol-4-yl)-2-methyl-propan-2-ol
1-(2-[5-(2-Amino-pyridin-4-yl)-1H-thieno[3,2-c]pyrazol-3-yl]-6-{[methyl-(1,2,2-trimethyl-propyl)-amino]-methyl}-1H-indol-4-yl)-2-methyl-propan-2-ol
4-[3-(4-(2-Hydroxy-2-methyl-propyl)-6-{[methyl-(1,2,2-trimethyl-propyl)-amino]-methyl}-1H-indol-2-yl)-1H-thieno[3,2-c]pyrazol-5-yl]-1H-pyridin-2-one
1-{6-(2,2-Dimethyl-morpholin-4-ylmethyl)-2-[5-(1H-pyrazol-4-yl)-1H-thieno[3,2-c]pyrazol-3-yl]-1H-benzoimidazol-4-yl}-2-methyl-propan-2-ol
2-Methyl-1-{6-{[methyl-(1,2,2-trimethyl-propyl)-amino]-methyl}-2-[5-(2H-[1,2,3]triazol-4-yl)-1H-thieno[3,2-c]pyrazol-3-yl]-1H-benzoimidazol-4-yl}propan-2-ol and
1-(2-[5-(2-Amino-pyridin-4-yl)-1H-thieno[3,2-c]pyrazol-3-yl]-6-{[methyl-(1,2,2-trimethyl-propyl)-amino]-methyl}-1H-benzoimidazol-4-yl)-2-methyl-propan-2-ol
or the pharmaceutically acceptable salts thereof.

5. A method of treating a disease or condition chosen from chronic inflammation, allergies, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease, and lupus erythematosus comprising administering to a patient a pharmaceutically effective amount of a compound according to any one of claims 1-4.

6. A method of treating a disease or condition chosen from asthma, chronic obstructive pulmonary disease (COPD), adult respiratory distress syndrome (ARDS), bronchitis, conjunctivitis, dermatitis and allergic rhinitis comprising administering to a patient a pharmaceutically effective amount of a compound according to any one of claims 1-4.

7. A pharmaceutical composition comprising a pharmaceutically effective amount of a compound according to any one of claims 1-4 and one or more pharmaceutically acceptable carriers and/or adjuvants.
